(19) United States
(12) Patent Application Publication
(10) Pub. No.: US 2002/0110701 A1
(43) Pub. Date: Aug. 15, 2002

(54) ELECTROLUMINESCENT ASSEMBLIES USING BLEND SYSTEMS

(76) Inventors: ROLF WEHRMANN, KREFELD (DE); HELMUT WERNER HEUER, KREFELD (DE); FRIEDRICH JONAS, AACHEN (DE); ANDREAS ELSCHNER, MULHEIM (DE); ANDREA MAYER, BROOKLYN, NY (US); MARTIN HUPPAUFF, STUTTGART (DE); HARTWIG ANDRIES, RUPELMONDE (BE)

Correspondence Address: CONNOLLY BOVE LODGE & HUTZ, LLP, 1220 N MARKET STREET, P O BOX 2207, WILMINGTON, DE, 19899

(21) Appl. No.: 09/267,441

(22) Filed: Mar. 12, 1999

(30) Foreign Application Priority Data
Mar. 20, 1998 (DE) 198 12 258.6

(51) Int. Cl. H05B 33/12

(52) U.S. Cl. 428/690; 428/917; 313/504; 313/506

(57) ABSTRACT

Electroluminescent assemblies containing a substrate, an anode, an electroluminescent element and a cathode, where at least one of the two electrodes is transparent or semitransparent in the visible spectral region and the electroluminescent element can contain in order:

a hole injection zone, a hole transport zone, an electroluminescent zone, an electron transport zone and an electron injection zone, characterized in that the hole injection zone contains an uncharged or cationic polythiophene of the formula (I),

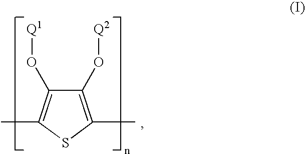

where

Q¹ and Q² represent, independently of one another, hydrogen, substituted or unsubstituted (C₁-C₂₀)-alkyl, CH₂OH or (C₆-C₁₄)-aryl or

Q¹ and Q² together represent —(CH₂)ₘ—CH₂— where m=0 to 12, preferably 1 to 5, (C₆-C₁₄)-arylene, and

n represents an integer from 2 to 10,000, preferably from 5 to 5000,

and the hole transport zone adjoining the hole injection zone contains one or more aromatic amine compounds.

An electroluminescent (EL) assembly is characterized in that it emits light with a flow of current on application of an electric potential. Such assemblies have long been known in industry under the name light emitting diodes (LEDs). The emission of light occurs as a result of positive charges (holes) and negative charges (electrons) recombining with emission of light.

In the development of light-emitting components for electronics or optics, use is at present made mainly of inorganic semiconductors such as gallium arsenide. On the basis of such substances, display elements in the form of dots can be produced. Large-area assemblies are not possible.

Apart from the semiconductor light-emitting diodes, electroluminescent assemblies based on vapour-deposited low molecular weight organic compounds are known (U.S. Pat. Nos. 4,539,507, 4,769,262, 5,077,142, EP-A 406 762, EP-A 278 758, EP-A 278 757).

Furthermore, polymers such as poly-(p-phenylenes) and poly-(p-phenylenevinylenes) (PPVs) have been described as electroluminescent polymers: G. Leising et al., Adv. Mater. 4 (1992) No. 1; Friend et al., J. Chem. Soc., Chem. Commun. 32 (1992); Saito et al., Polymer, 1990, Vol. 31, 1137; Friend et al., Physical Review B, Vol. 42, No. 18, 11670 or WO-A 90/13148. Further examples of PPVs in electroluminescent displays are described in EP-A 443 861, WO-A 92/03490 and WO-A 92/003491.

EP-A 0 294 061 discloses an optical modulator based on polyacetylene.

To produce flexible polymer LEDs, Heeger et al. have proposed soluble conjugated PPV derivatives (WO-A 92/16023).

Polymer blends of different compositions are likewise known: M. Stolka et al., Pure & Appt. Chem., Vol. 67, No. 1, pp 175-182, 1995; H. Bässler et al., Adv. Mater. 1995, 7, No. 6, 551; K. Nagai et al., Appl. Phys. Lett. 67 (16), 1995, 2281; EP-A 532 798.

The organic EL assemblies generally contain one or more layers of organic charge transport compounds. The in-principle structure in order of the layers is as follows:

| | |
|---|---|
| 1 | Support, substrate |
| 2 | Base electrode/anode |
| 3 | Hole injection layer |
| 4 | Hole transport layer |
| 5 | Light-emitting layer |
| 6 | Electron transport layer |
| 7 | Electron injection layer |
| 8 | Top electrode/cathode |
| 9 | Contacts |
| 10 | Sheathing, encapsulation. |

The layers 3 to 7 represent the electroluminescent element.

This structure represents the most general case and can be simplified by leaving out individual layers so that one layer assumes a plurality of tasks. In the simplest case, an EL assembly consists of two electrodes between which there is located an organic layer which fulfils all functions including the emission of light. Such systems are described, for example, in the application WO-A 90 13148 on the basis of poly-(p-phenylene-vinylene).

Multilayer systems can be built up by vapour deposition processes in which the layers are applied successively from the gas phase or by casting processes. Owing to the higher process speeds, casting processes are preferred. However, the partial dissolution of a layer which has already been applied when it is covered by the next layer can in certain cases present a difficulty.

The object of the present invention is to provide electroluminescent assemblies having a high light flux, where the mixture to be applied can be applied by casting.

It has been found that electroluminescent assemblies containing the blend system described below meet these requirements. In the following, the term “zone” is equivalent to “layer”.

The present invention accordingly provides electroluminescent assemblies containing a substrate, an anode, an electroluminescent element and a cathode, where at least one of the two electrodes is transparent or semitransparent in the visible spectral region and the electroluminescent element can contain in order,

a hole injection zone, a hole transport zone, an electroluminescent zone, an electron transport zone and an electron injection zone, characterized in that the hole injection zone contains an uncharged or cationic polythiophene of the formula (I),

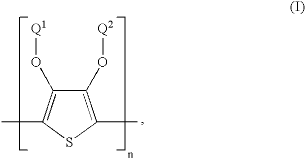

where

Q¹ and Q² represent, independently of one another, hydrogen, substituted or unsubstituted (C₁-C₂₀)-alkyl, CH₂OH or (C₆-C₁₄)-aryl or

Q¹ and Q² together represent —(CH₂)ₘ—CH₂— where m=0 to 12, preferably 1 to 5, (C₆-C₁₄)-arylene, and

n represents an integer from 2 to 10,000, preferably from 5 to 5000,

and the hole transport zone adjoining the hole injection zone contains one or more aromatic amine compounds, preferably substituted or unsubstituted triphenylamine compounds, particularly preferably 1,3,5-tris(aminophenyl)benzene compounds A of the formula (II).

The zones or zone located between hole injection zone and cathode can also assume a plurality of functions, i.e. one zone can, for example, contain hole transport, electroluminescent, electron transport and/or electron injection substances.

The electroluminescent element can also contain one or more transparent polymeric binders B.

The substituted or unsubstituted 1,3,5-tris(aminophenyl)benzene compound A represents an aromatic tertiary amino compound of the general formula (II)

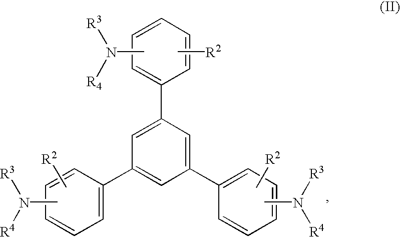

in which

R² represents hydrogen, substituted or unsubstituted alkyl or halogen, R³ and R⁴ represent, independently of one another, substituted or unsubstituted (C₁-C₁₀)-alkyl, (C₁-C₁₀)-alkoxy, alkoxycarbonyl-substituted (C₁-C₁₀)-alkyl, in each case substituted or unsubstituted aryl, aralkyl or cycloalkyl.

R³ and R⁴ preferably represent, independently of one another, (C₁-C₆)-alkyl, in particular methyl, ethyl, n- or isopropyl, n-, iso-, sec- or tert-butyl, (C₁-C₄)-alkoxycarbonyl-(C₁-C₆)-alkyl, for example methoxy-, ethoxy-, propoxy-, butoxy-carbonyl-(C₁-C₄)-alkyl, in each case unsubstituted or (C₁-C₄)-alkyl- and/or (C₁-C₄)-alkoxy-substituted phenyl-(C₁-C₄)-alkyl, naphthyl-(C₁-C₄)-alkyl, cyclopentyl, cyclohexyl, phenyl, naphthyl or anthracyl.

Particularly preferably R³ and R⁴ represent, independently of one another, unsubstituted phenyl or naphthyl or in each case singly to triply methyl-, ethyl-, n-, iso-propyl-, methoxy-, ethoxy-, n- and/or iso-propoxy-substituted phenyl or naphthyl.

R² preferably represents hydrogen, (C₁-C₆)-alkyl such as methyl, ethyl, n- or iso-propyl, n-, iso-, sec- or tert-butyl, or halogen.

Such compounds and their preparation are described in U.S. Pat. No. 4,923,774 for use in electrophotography and this patent is hereby expressly incorporated by reference into the present description. The tris-nitrophenyl compound can, for example, be converted into the tris-aminophenyl compound by generally known catalytic hydrogenation, for example in the presence of Raney nickel (Houben-Weyl 4/1C, 14-102, Ullmann (4) 13, 135-148). The amino compound is reacted in a generally known manner with substituted halogenobenzenes.

Mention may be made, by way of example, of the following compounds, where the substitution on the phenyl ring can be ortho, meta and/or para to the amine nitrogen:

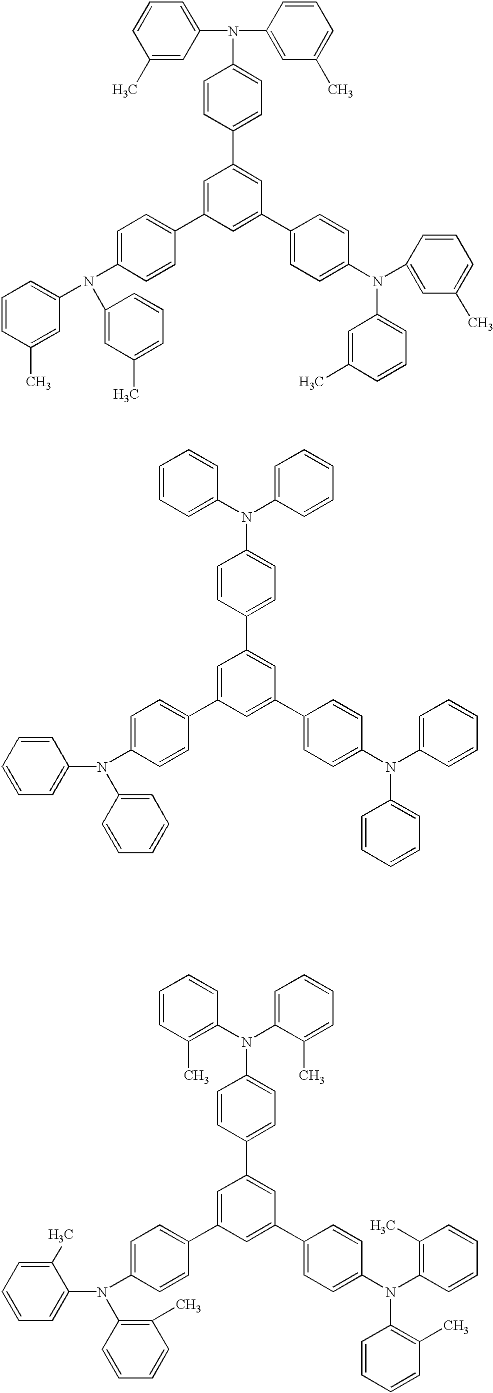

Apart from the component A, it is also possible to use, if desired, further hole conductors, e.g. in the form of a mixture with the component A, for constructing the electroluminescent element. Use can here be made either of one or more compounds of the formula (II), including mixtures of isomers, or mixtures of hole transport compounds with compounds of A, having the general formula (II), of various structures.

A listing of further possible hole conductor materials is given in EP-A 532 798.

In the case of mixtures of aromatic amines, the compounds can be used in any ratio.

Examples which may be mentioned are:

Anthracene compounds, e.g. 2,6,9,10-tetraisopropoxyanthracene; oxadiazole compounds, e.g. 2,5-bis(4-diethylaminophenyl)-1,3,4-oxadiazole, triphenylamine compounds, e.g. N,N′-diphenyl-N,N′-di(3-methylphenyl)-1,1′-biphenyl-4,4′-diamine; aromatic tertiary amines, e.g. N-phenylcarbazole, N-isopropyl-carbazole and compounds which can be used in hole transport layers, as are described in the Japanese Patent Application JP-A 62-264 692; also pyrazoline compounds, e.g. 1-phenyl-3-(p-diethylaminostyryl) -5-(p-diethylaminophenyl)-2-pyrazoline; styryl compounds, e.g. 9-(p-diethylaminostyryl)-anthrazene; hydrazone compounds, e.g. bis-(4-dimethylamino -2-methylphenyl)-phenyl-methane; stilbene compounds, e.g. -(4-methoxyphenyl) -4-N,N-diphenylamino-(4′-methoxy)stilbene, enamine compounds, e.g. 1,1-(4,4′-diethoxyphenyl)-N,N-(4,4′-dimethoxyphenyl)enamine; metal or nonmetal phthalocyanines and porphyrin compounds.

Preference is given to triphenylamine compounds and/or aromatic tertiary amines, with the compounds mentioned by way of example being particularly preferred.

Materials which have hole conductor properties and can be used in pure form or as mixing partners for component A are, for example, the following compounds, where

X¹ to X⁶ represent, independently of one another, H, halogen, alkyl, aryl, alkoxy, aryloxy.

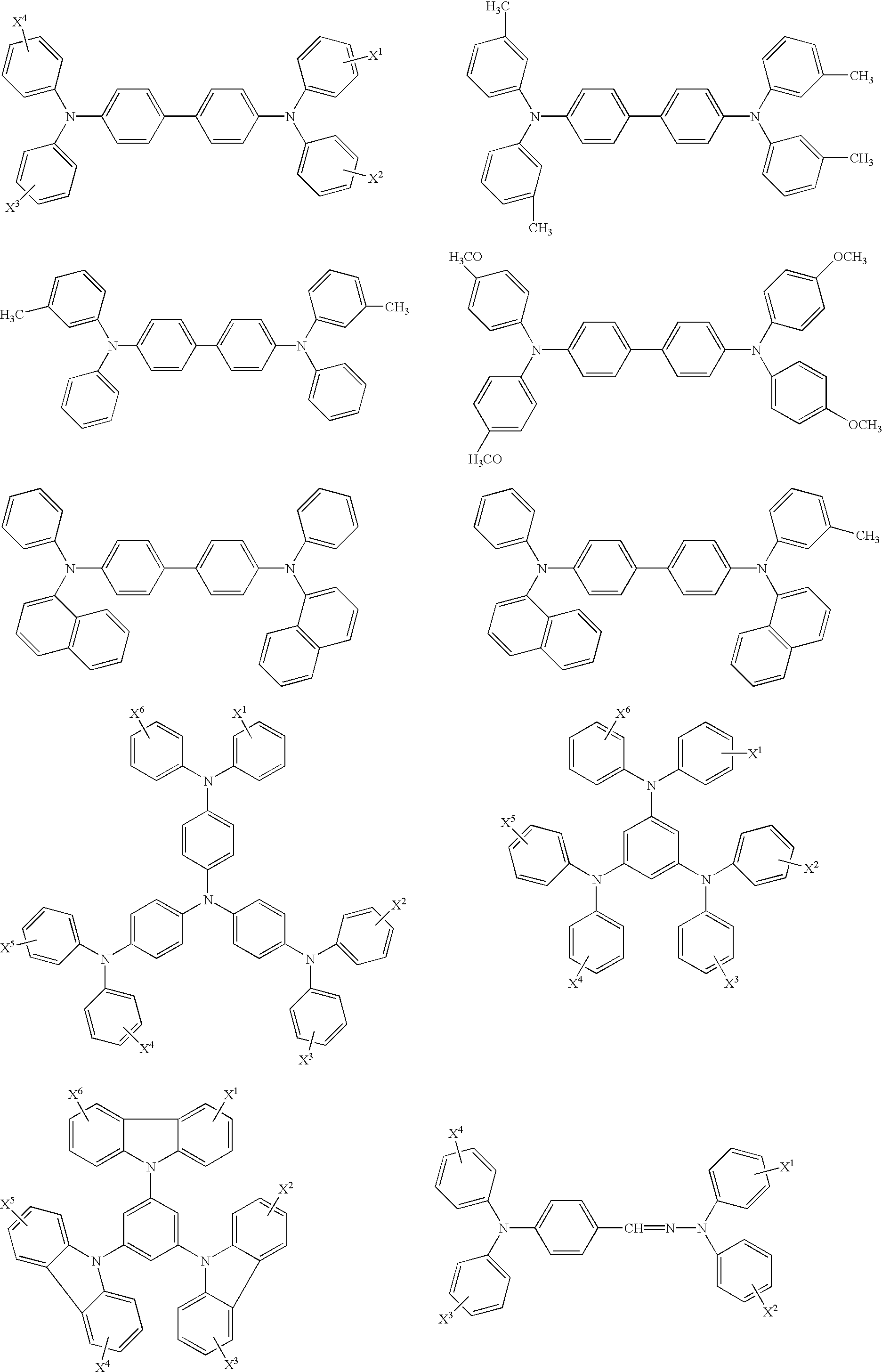

These and further examples are described in J. Phys. Chem. 1993, 97, 6240-6248 and Appl. Phys. Lett., Vol. 66, No. 20, 2679-2681.

In general, various amines having different base structures and/or different substitution patterns can be mixed.

X¹ to X⁶ prefererably represent, independently of one another, hydrogen, fluorine, chlorine, bromine, (C₁-C₁₀)-, in particular (C₁-C₄)-alkyl or -alkoxy, phenyl, naphthyl, phenoxy and/or naphthyloxy. The aromatic rings can be monosubstituted, disubstituted, trisubstituted or tetrasubstituted by one or more, identical or different radicals X¹ to X⁶.

The polythiophenes of the structural repeating unit of the formula (I) are known (cf. EP-A 440 958 and 339 340). The preparation of the dispersions or solutions according to the invention is described in EP-A 440 957 and DE-A 4 211 459.

The polythiophenes in the dispersion or solution are preferably used in cationic form as is obtained, for example, by treatment of the uncharged thiophenes with oxidizing agents. Customary oxidizing agents such as potassium peroxodisulphate are used for the oxidation. The oxidation gives the polythiophenes positive charges which are not shown in the formulae since their number and their position cannot be determined definitively. According to EP-A 339 340, they can be prepared directly on supports.

Q¹ and Q² in formula (I) preferably represent —(CH₂)ₘ—CH₂— where m =1 to 4, very particularly preferably ethylene.

Preferred cationic or uncharged polydioxythiophenes are built up of structural units of the formula (Ia) or (Ib)

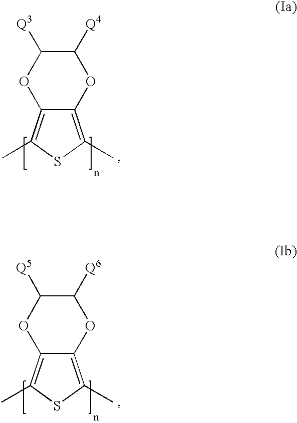

where

Q³ and Q⁴ represent, independently of one another, hydrogen, substituted or unsubstituted (C₁-C₁₈)-alkyl, preferably (C₁-C₁₀)-, in particular (C₁-C₆)-alkyl, (C₂-C₁₂)-alkenyl, preferably (C₂-C₈)-alkenyl, (C₃-C₇)-cycloalkyl, preferably cyclopentyl, cyclohexyl, (C₇-C₁₅)-aralkyl, preferably phenyl-(C₁-C₄)-alkyl, (C₆-C₁₀)-aryl, preferably phenyl, naphthyl, (C₁-C₁₀)-alkoxy, preferably (C¹-C₁₀)-alkoxy, for example methoxy, ethoxy, n- or iso-propoxy, or (C₂-C₁₈)-alkyloxy ester and

Q⁵ and Q⁶ represent, independently of one another, hydrogen, but not both simultaneously, or (C₁-C₁₈)-alkyl, preferably (C₁-C₁₀)-, in particular (C₁-C₆)-alkyl, (C₂-C₁₂)-alkenyl, preferably (C₂-C₈)-alkenyl, (C₃-C₇)-cycloalkyl, preferably cyclopentyl, cyclohexyl, (C₇-C₁₅)-aralkyl, preferably phenyl-(C₁-C₄)-alkyl, (C₆-C₁₀)-aryl, preferably phenyl, naphthyl, (C₁-C₁₈)-alkoxy, preferably (C₁-C₁₀)-alkoxy, for example methoxy, ethoxy, n- or iso-propoxy, or (C₂-C₁₈)-alkyloxy ester which are each substituted by at least one sulphonate group, where if Q⁵ is hydrogen, Q⁶ is different from hydrogen and vice versa,

n represents an integer from 2 to 10,000, preferably from 5 to 5000.

Particular preference is given to cationic or uncharged polythiophenes of the formulae (Ia-1) and (Ib-1)

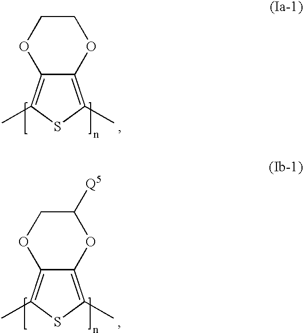

where

Q⁵ and n are as defined above.

As polyanions, use is made of the anions of polymeric carboxylic acids such as polyacrylic acids, polymethacrylic acid or polymaleic acids and polymeric sulphonic acids such as polystyrenesulphonic acids and polyvinylsulphonic acids. These polycarboxylic and polysulphonic acids can also be copolymers of vinylcarboxylic and vinylsulphonic acids with other polymerizable monomers such as acrylic esters and styrene.

Particular preference is given to the anion of polystyrenesulphonic acid as counterion.

The molecular weight of the polyacids forming the polyanions is preferably from 1000 to 2,000,000, particularly preferably from 2000 to 500,000. The polyacids or their alkali metal salts are commercially available, e.g. polystyrenesulphonic acids and polyacrylic acids, or else can be prepared by known methods (see, for example, Houben-Weyl, Methoden der organischen Chemie, Volume E 20, Makromolekulare Stoffe, Part 2 (1987), p. 1141 ff).

In place of the free polyacids required for formation of the dispersions of polydioxythiophenes and polyanions, it is also possible to use mixtures of alkali metal salts of the polyacids and appropriate amounts of monoacids.

In the case of the formulae (Ib) and (Ib-1), the polydioxythiophenes bear positive and negative charges in the monomer unit itself.

The electroluminescent element can, if desired, contain a further functionalized compound selected from the group consisting of hole injection and/or hole transport materials, a luminescent material C and, if desired, electron transport materials, where the hole transport zone can contain, apart from the component A, one or more further hole transport, electroluminescent, electron transport and/or electron injection compounds, where at least one zone is present, individual zones can be left out and the zone(s) present can assume a plurality of functions.

As luminescent material (component C), it is possible to use substances which display photoluminescence, i.e. fluorescent and laser dyes, but also metal complexes and chelates or inorganic nanosize particles.

Examples of fluorescent and laser dyes are stilbenes, distilbenes, methine dyes, coumarins, naphthalimides, perylenes, rubrene, quinacridones, phenanthrenes, anthracenes, phthalocyanines, etc. Further examples are described in EP-A 532 798.

In the case of the metal complexes, it is possible to employ monovalent, divalent or trivalent metals in general which are known to form chelates.

The metal can be a monovalent, divalent or trivalent metal, for example lithium, sodium, potassium, magnesium, calcium, boron, aluminium, gallium, indium, rare earths. Suitable inorganic nanosize particles are, for example, semiconductors such as CdS, CdSe, ZnS or ZnO.

Suitable examples of component C) are the oxine complexes (8-hydroxyquinoline complexes) of Al³⁺, Mg²⁺, In³⁺, Ga³⁺, Zn²⁺, Be²⁺, Li⁺, Ca²⁺, Na⁺ or tris(5-methyloxine)aluminium and tris(5-chloro-quinoline)gallium. Complexes with rare earth metals can also be used.

Examples of metal complexes are

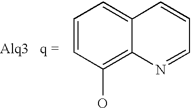

Inq₃, Gaq₃, Znq₂, Beq₂, Mgq₂,

or Al(qa)₃, Ga(qa)₃, In(qa)₃, Zn(qa)₂, Be(qa)₂, Mg(qa)₂, where

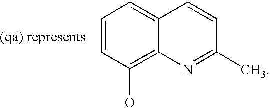

It is also possible to use metal complexes which bear different groups. Examples of such compounds are Alq₂ OR, Gaq₂ OR, Al(qa)₂ OR, Ga(qa)₂ OR, Alqa₂ OCOR, Ga(qa)₂ OCOR, Alq₂X and Gaq₂X and also Al(qa)₂X and Ga(qa)₂X, where X represents halogen and R represents in each case substituted or unsubstituted alkyl, aryl, arylalkyl and cycloalkyl, preferably in each case unsubstituted or halogen- and/or cyano-substituted (C₁-C₁₂)-alkyl, in particular (C₁-C₈)-alkyl, (C₄-C₈)-cycloalkyl, in particular cyclopentyl or cyclohexyl, aryl having from 6 to 20 carbon atoms, in particular phenyl, naphthyl, phenyl-(C₁-C₄)-alkyl, where the cyclic and aromatic radicals can also be (C₁-C₄)-alkyl-substituted.

The carbon chains are in each case linear or branched.

A listing of suitable metal complexes and electron transporting compounds is given in EP-A 525 739, EP-A 579 151 and EP-A 757 088. Preparative methods are described, for example, in U.S. Pat. No. 4,769,292.

It is also possible to use mixtures of different metal complexes.

As binders B), it is possible to use polymers and/or copolymers such as polycarbonates, polyester carbonates, polystyrene, poly-α-methylstyrene, copolymers of styrene such as SAN or styrene-acrylates, polysulphones, polymers based on monomers containing vinyl groups, e.g. poly(meth)acrylates, polyvinylpyrrolidone, polyvinylcarbazole, vinyl acetate and vinyl alcohol polymers and copolymers, polyolefins, cyclic olefin copolymers, phenoxy resins, etc. Mixtures of various polymers can also be used. The polymeric binders B) have molecular weights of from 1000 to 200,000 g/mol, are soluble and film-forming and are transparent in the visible spectral region. They are described, for example, in Encyclopedia of Polymer Science and Engineering, 2^{nd} Ed., Wiley-Interscience.

The components can, however, also be located in separate layers.

The individual zones of the electroluminescent element can either be deposited from a solution or from the gas phase or a combination of both methods.

To produce the layer structure, the components are, for example, dissolved in a suitable solvent and applied to a suitable substrate by casting, doctor blade coating, printing or spin-coating. The substrate can be, for example, glass or a plastic material which is provided with a, possibly transparent, electrode. The plastic material can be, for example, a film of polycarbonate, polyester such as polyethylene terephthalate or polyethylene naphthalate, polysulphone or polyimide.

Suitable transparent and semitransparent electrodes are

a) metal oxides, e.g. indium-tin oxide (ITO), tin oxide (NESA), zinc oxide, doped tin oxide, doped zinc oxide, etc.,

b) semitransparent metal films, e.g. Au, Pt, Ag, Cu etc.,

c) conductive polymer films such as polyanilines, polythiophenes, etc.

The metal oxide electrodes and the semitransparent metal film electrodes are applied in a thin layer by techniques such as vapour deposition, sputtering, platination, etc. The conductive polymer films are applied from solution by techniques such as spin coating, casting, doctor blade coating, printing, etc.

The thickness of the transparent or semitransparent electrode is from 3 nm to a number of μm, preferably from 10 nm to 500 nm.

In a preferred assembly, the electroluminescent element is applied directly to the anode. In an alternative embodiment, the electroluminescent element can be applied to the support provided with a cathode.

The thickness of the electroluminescent element is generally from 10 nm to 5 μm, preferably from 20 nm to 1 μm, particularly preferably from 50 nm to 600 nm.

A listing of suitable intermediate charge transport layers, which may be hole transporting and/or electron transporting materials which can be in polymeric or low molecular weight form, if desired as a blend, is given in EP-A 532 798.

The content of low molecular weight compounds, viz. hole injection, hole transport, electroluminescent, electron transporting and electron injection substances, in a polymeric binder can generally be varied in the range from 2 to 97% by weight; the content is preferably from 5 to 95% by weight, particularly preferably from 10 to 90% by weight, in particular from 10 to 85% by weight.

Film-forming low molecular weight compounds can also be used in pure form (100% pure).

Blends which consist exclusively of low molecular weight compounds can be deposited from the gas phase; soluble and film-forming blends which may (though not necessarily) contain a binder B) in addition to low molecular weight compounds can be deposited from solution, e.g. by means of spin coating, casting, doctor blade coating, printing.

It is also possible to apply emitting and/or electron transporting substances in a separate layer on the hole conductor layer containing the component A. An emitting substance can also be added as dopant to the layer containing the compound A and an electron transporting substance can be additionally applied. An electroluminescent substance can also be added to the electron injection or electron conductor layer.

The content of low molecular weight electron transporting in the polymeric binder can be varied in the range from 2 to 95% by weight; the content is preferably from 5 to 90% by weight, particularly preferably from 10 to 85% by weight. Film-forming electron conductors can also be used in pure form (100% pure).

The counterelectrode to the electrode located on the support comprises a conductive substance which may be transparent and preferably contains metals, e.g. Ca, Al, Au, Ag, Mg, In etc., or alloys and metal oxides, conductive polymers which can be applied by techniques such as vapour deposition, sputtering, platination, printing, casting or doctor blade coating.

The assembly of the invention is brought into contact with the two electrodes by two electric leads (e.g. metal wires).

On application of a DC voltage in the range from 0.1 to 100 volt, the assemblies emit light having a wavelength of from 200 to 2000 nm.

The assemblies of the invention are suitable for producing units for lighting and for display of information.

### EXAMPLES

### Example 1

In the construction according to the invention of an organic light-emitting diode (OLED), the following procedure is employed:

1. Cleaning of the ITO substrate

ITO-coated glass (Merck Balzers AG, Principality of Lichtenstein, Part No. 253 674 XO) is cut into 50 mm×50 mm pieces (substrates). The substrates are subsequently cleaned for 15 minutes in 3% strength aqueous Mukasol solution in an ultrasonic bath. The substrates are then rinsed with distilled water and spun dry in a centrifuge. This rinsing and drying procedure is repeated 10 times.

2. Application of the Baytron® P layer to the ITO

About 10 ml of the about 1.2% strength poly(ethylenedioxythiophene)-polysulphonic acid solution (BAYER AG, Leverkusen, Germany, Baytron® P) are filtered (Millipore HV, 0.45 μm). The substrate is subsequently placed on a spin coater and the filtered solution is distributed on the ITO-coated side of the substrate. The supernatant solution is subsequently spun off by rotation of the plate at 500 rpm for a period of 3 minutes. The substrate which has been coated in this way is then dried on a hotplate for 5 minutes at 110° C. The thickness of the layer is 60 nm (Tencor, Alphastep 200).

3. Application of the hole transporting layer

5 ml of a 1.5% strength dichloroethane solution of 1 part by weight of polyvinylcarbazole (BASF, Ludwigshafen, Germany, Luvican) and 2 parts by weight of the amine A are filtered (Millipore HV, 0.45 μm) and distributed on the dried Baytron P layer. The supernatant solution is subsequently spun off by rotation of the plate at 800 rpm for 50 seconds. The substrate which has been coated in this way is then dried for 5 minutes at 110° C. on a hotplate. The total layer thickness is 150 mm.

4. Application of the light-emitting/electron injection layer

5 ml of a 1.5% strength methanol solution of the metal complex 1 are filtered (Millipore HV, 0.45 μm) and distributed on the dried hole conductor layer. The supernatant solution is subsequently spun off by rotation of the plate at 400 rpm for 30 seconds. The substrate which has been coated in this way is then dried for 5 minutes at 110° C. on a hotplate. The total layer thickness is 200 nm.

5. Vapour deposition of the metal cathode

A metal electrode is vapour-deposited onto the organic layer system. For this purpose, the substrate is placed, with the organic layer system downwards, on a perforated mask (hole diameter 5 mm). From two vapour deposition boats, the elements Mg and Ag are vaporized in parallel at a pressure of 10⁻³ Pa. The vapour deposition rates are 28 Å/sec for Mg and 2 Å/sec for Ag. The thickness of the vapour-deposited metal contacts is 500 nm.

The two electrodes of the organic LED are connected via electric leads to a voltage source. The positive pole is connected to the ITO electrode and the negative pole is connected to the Mg/Ag electrode.

From a voltage of only 3 volt, electroluminescence can be detected by means of a photodiode (EG & G C30809E). At a voltage of 10 volt, the current per unit area is 35 mA/cm² and the electroluminescence is readily visible. The colour of the electroluminescence is green-blue.

### Example 2

The procedure for the construction according to the invention of an OLED is as in Example 1 with the following difference:

5 ml of a 1.0% strength methanol solution of 1 part by weight of metal complex 1 and 0.02 part by weight of fluorescent dye F are filtered (Millipore HV, 0.45 μm) and distributed on the dried hole transporting layer. The supernatant solution is subsequently spun off by rotation of the plate at 400 rpm for 30 seconds. The substrate which has been coated in this way is subsequently dried for 5 minutes at 110° C. on a hotplate.

From a voltage of 3 volt, electroluminescence can be detected by means of a photodiode (EG & G C30809E). At a voltage of 10 volt, the current per unit area is 180 mA/cm² and the electroluminescence is readily visible. The colour of the electroluminescence is green-blue.

### Example 3

The procedure for the construction according to the invention of an OLED is as in Example 1 with the following difference:

5 ml of a 1.0% strength methanol solution of metal complex 2 are filtered (Millipore HV, 0.45 μm) and distributed on the dried hole transporting layer. The supernatant solution is subsequently spun off by rotation of the plate at 250 rpm for 40 seconds. The substrate which has been coated in this way is subsequently dried for 5 minutes at 110° C. on a hotplate.

From a voltage of 3 volt, electroluminescence can be detected by means of a photodiode (EG & G C30809E). At a voltage of 10 volt, the current per unit area is 100 mA/cm² and the electroluminescence is readily visible. The colour of the electroluminescence is green-blue.

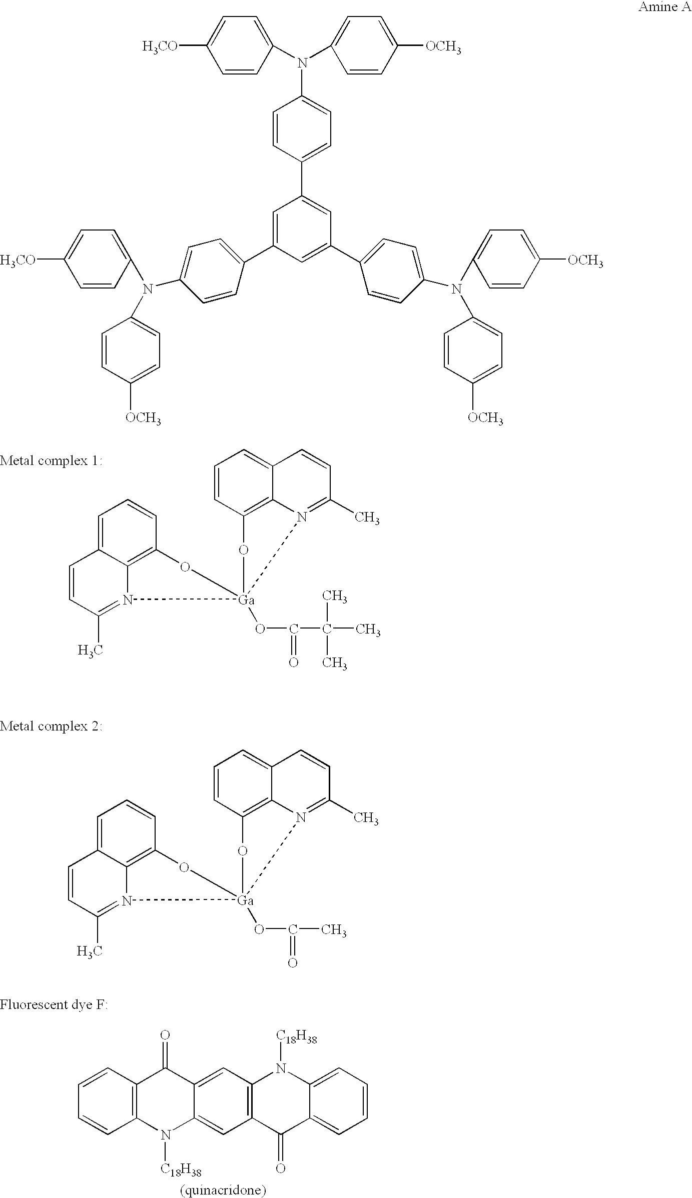

### Example 4

Substrate: Baltracon 255 (Balzers)

Hole injection layer Baytron®P (Bayer AG, Leverkusen) about 1.2% strength solution, solvent H₂O, applied at 800 rpm, layer thickness about 70 nm.

Hole transporting layer: polystyrene from Aldrich, 89555 Steinheim, Germany, Catalogue No. 18, 242-7) (PS)+amine A (1:1) from dichloroethane solvent, 1% strength solution applied at 800 rpm, layer thickness about 60 nm.

Electroconductive layer: Alq₃ vapour-deposited, about 60 nrm at a pressure of 10⁻⁶ mbar. Polystyrene from Aldrich, 89555 Steinheim, Germany, Catalogue No. 18, 242-7)

Cathode: Mg/Ag (10:1), vapour-deposited (codeposition), layer thickness about 200 nm.

At a voltage of 9 volt, the current is 21 mA/cm²; the light intensity is 290 Cd/m².

### Example 5

Substrate: Baltracon 255 (Balzers)

Hole injection layer: Baytron® P (Bayer AG, Leverkusen), about 1.2% strength solution, solvent H₂O, applied at 800 rpm, layer thickness about 70 nm.

Hole transporting layer: PS+amine A (1:2) from a cyclohexane/THF (tetrahydrofuran) solvent mixture (1:10), 1% strength solution applied at 800 rpm, layer thickness about 60 nm.

Electron transporting layer: metal complex 3, 1% strength from methanol applied at 300 rpm, layer thickness about 30 nm.

Cathode: Mg/Ag (10:1), vapour-deposited (codeposition), layer thickness about 200 nm. At a voltage of 7 volt, the current is 17 mA/cm²; the light intensity is 210 Cd/m².

Light emission: blue-green.

### Example 6

Substrate: Baltracon 255 (Balzers)

Hole injection layer: Baytron® P (Bayer AG, Leverkusen) about 1.2% strength solution, solvent H₂O, applied at 800 rpm, layer thickness about 70 nm.

Hole transporting layer: polyvinylcarbazole +amine A (1:4) doped with rubrene, 2.5% based on solids content from dichloroethane solvent, 1% strength solution applied at 800 rpm, layer thickness about 60 nm (polyvinylcarbazole =Luvican EP, BASF AG, Ludwigshafen, Germany).

Electroconductive layer: Alq₃, vapour-deposited, about 60 nm at a pressure of 10-6 mbar.

Cathode: Mg/Ag (10:1), vapour-deposited (codeposition), layer thickness about 200 nm.

Light emission: yellow

At a voltage of 9 volt, the current is 54 mA/cm²; the light intensity is 1200 Cd/m².

### Example 7

Substrate: Baltracon 255 (Balzers)

Hole injection layer: Baytron®P (Bayer AG, Leverkusen), about 1.2% strength solution, solvent H₂O, applied at 800 rpm, layer thickness about 70 nm.

Hole transporting layer: PS +amine A (1:2) from cyclohexane/THF solvent mixture (1:10), 1% strength solution applied at 800 rpm, layer thickness about 60 nm.

Electron transporting layer: metal complex 3, 1% strength from methanol applied at 300 rpm, layer thickness about 30 nm.

Cathode: Mg/Ag (10:1), vapour-deposited (codeposition), layer thickness about 200 nm.

At a voltage of 9 volt, the current is 21 mA/cm²; the light intensity is 212 Cd//m².

Light emission: blue-green

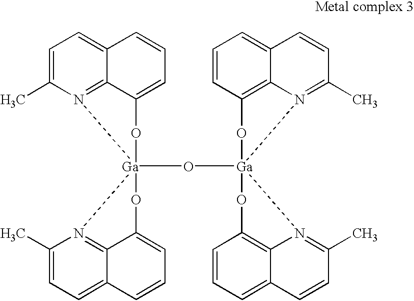

### Example 8

Substrate: Baltracon 255 (Balzers)

Hole injection layer: Baytron®P (Bayer AG, Leverkusen) about 1% strength solution, solvent H₂O, applied at 800 rpm, layer thickness about 70 nm.

Hole transporting layer: polyvinylcarbazole +amine A (1:2) from cyclohexane/THF solvent mixture (1:10), 1% strength solution applied at 800 rpm, layer thickness about 60 nm.

Electron transporting layer: Alq₃, vapour-deposited, about 60 nm at a pressure of 10-6 mbar.

Cathode: Mg/Ag (10:1), vapour-deposited (codeposition), layer thickness about 200 nm.

At a voltage of 8 volt, the current is 30 mA/cm²; the light intensity is 500 Cd/m².

### Example 9

Substrate: Baltracon 255 (Balzers)

Hole injection layer: Baytron®P (Bayer AG, Leverkusen), about 1.2% strength solution, solvent H₂O, applied at 800 rpm, layer thickness about 70 nm.

Hole transporting layer: polyvinylcarbazole +amine B (1:1) from dichloroethane solvent, 1% strength solution, applied at 800 rpm, layer thickness about 60 nm.

Electron transporting layer: Alq₃, vapour-deposited, about 60 nm at a pressure of 10⁻⁶mbar.

Cathode: Mg/Ag (10:1), vapour-deposited (codeposition), layer thickness about 200 nm.

At a voltage of 14 volt, the current is 55 mA/cm²; the light intensity is 521 Cd/m².

### Example 10

Substrate: Baltracon 255 (Balzers)

Hole injection layer: Baytron®P (Bayer AG, Leverkusen), about 1.2% strength solution, solvent H₂O, applied at 800 rpm, layer thickness about 70 nm. Hole transporting layer: polyvinylcarbazole +amine C (1:1) from dichloroethane solvent, 1% strength solution, applied at 800 rpm, layer thickness about 60 nm.

Electron transporting layer: metal complex 3, 1% from methanol applied at 300 rpm, layer thickness about 30 nm.

Cathode: Mg/Ag (10:1), vapour-deposited (codeposition), layer thickness about 200 nm.

At a voltage of 11 volt, the current is 29 mA/cm²; the light intensity is 315 Cd/m².

Light emission: blue-green

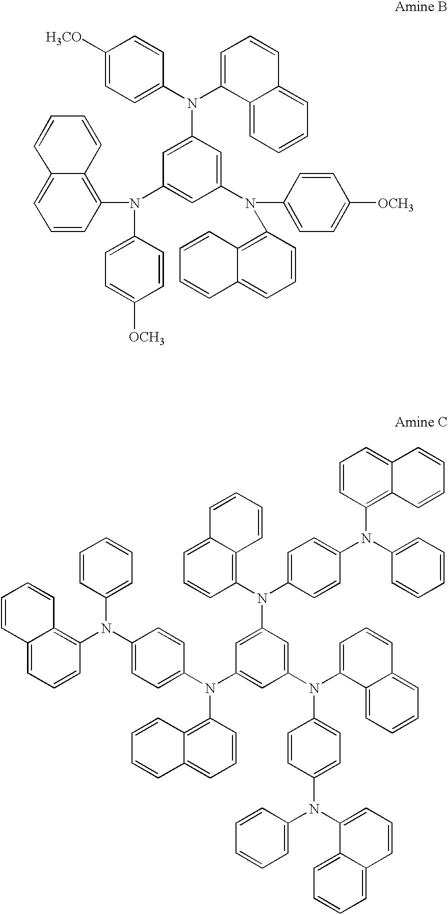

1. Electroluminescent assemblies containing a substrate, an anode, an electroluminescent element and a cathode, where at least one of the two electrodes is transparent or semitransparent in the visible spectral region and the electroluminescent element contains one or more zones selected from the group consisting of a hole injection zone, a hole transport zone, an electroluminescent zone, an electron transport zone and an electron injection zone in the specified order, where each of the zones present can also assume the function of the other zones, characterized in that the hole injection zone contains an uncharged or cationic polythiophene of the formula (I), 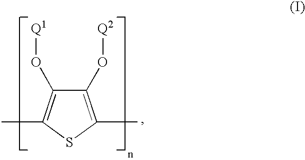
where
Q¹ and Q² represent, independently of one another, hydrogen, substituted or unsubstituted (C₁-C₂₀)-alkyl, CH₂OH or (C₆-C₁₄)-aryl or
Q¹ and Q² together represent —(CH₂)ₘ—CH₂— where m=0 to 12, preferably 1 to 5, (C₆-C₁₄)-arylene, and
n represents an integer from 2 to 10,000, preferably from 5 to 5000,
and the hole transport zone adjoining the hole injection zone contains one or more aromatic amine compounds A.

2. Electroluminescent assemblies according to claim 1, characterized in that one or more transparent polymeric binders B are present.

3. Electroluminescent assemblies according to claim 1, characterized in that at least one compound selected from among compounds of the general formula (II) 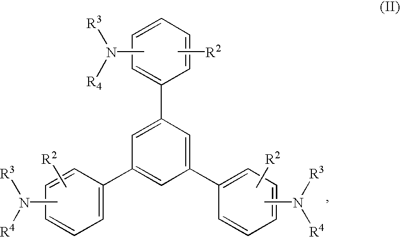
where
R² represents hydrogen, substituted or unsubstituted alkyl or halogen,
R³ and R⁴ represent, independently of one another, substituted or unsubstituted (C₁-C₁₀)-alkyl, (C₁-C₁₀)-alkoxy, alkoxycarbonyl-substituted (C₁-C₁₀)-alkyl, in each case substituted or unsubstituted aryl, aralkyl or cycloalkyl, is present as aromatic amine A.

4. Electroluminescent assemblies according to claim 3, characterized in that, in formula (II),
R³ and R⁴ represent, independently of one another, (C₁-C₆)-alkyl, (C₁-C₄)-alkoxycarbonyl-(C₁-C₆)-alkyl, in each case unsubstituted or (C₁-C₄)-alkyl- and/or (C₁-C₄)-alkoxy-substituted phenyl-(C₁-C₄)-alkyl, naphthyl-(C₁-C₄)-alkyl, cyclopentyl, cyclohexyl, phenyl, naphthyl or anthracyl and
R² represents hydrogen, (C₁-C₆)-alkyl or halogen.

5. Electroluminescent assemblies according to claim 1, characterized in that the aromatic amine A is selected from among the following compounds:

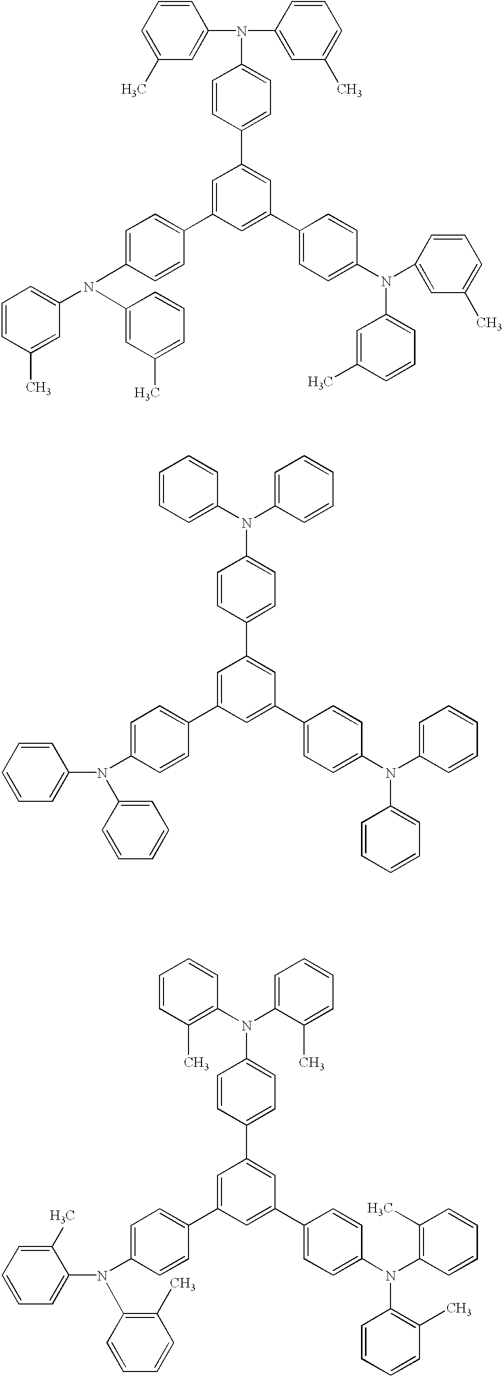

6. Electroluminescent assemblies according to claim 1, characterized in that further hole transporting materials which are different from the component A are present.

7. Electroluminescent assemblies according to claim 1, characterized in that the polythiophenes are built up of structural units of the formula (Ia) and/or (lb) 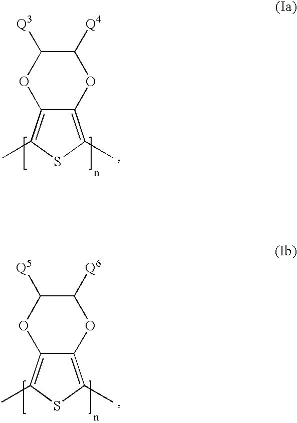
where
Q³ and Q⁴ represent, independently of one another, hydrogen, substituted or unsubstituted (C¹-C₁₈)-alkyl, (C₂-C₁₂)-alkenyl, (C₃-C₇)-cycloalkyl, (C₇-C₁₅)-aralkyl, (C₆-C₁₀)-aryl, (C₁-C₁₈)-alkoxy, or (C₂-C₁₈)-alkyloxy ester and
Q⁵ and Q⁶ represent, independently of one another, hydrogen, but not both simultaneously, or (C₁-C₁₈)-alkyl, (C₂-C₁₂)-alkenyl, (C₃-C₇)-cycloalkyl, (C₇-C₁₅)-aralkyl, (C₆-C₁₀)-aryl, (C¹-C₁₈)-alkoxy or (C₂-C₁₈)-alkyloxy ester which are each substituted by at least one sulphonate group,
n is an integer from 2 to 10,000.

8. Electroluminescent assemblies according to claim 1, characterized in that the polythiophenes are built up of structural units of the formulae (Ia-1) and/or (Ib-1) 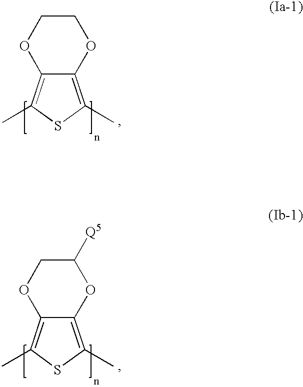
where
Q⁵ and n are as defined in claim 7.

9. Electroluminescent assemblies according to claim 1, characterized in that polyanions, preferably the anions of polymeric carboxylic acids and/or polymeric sulphonic acids, are present.

10. Electroluminescent assemblies according to claim 9, characterized in that polystyrenesulphonic acid or an alkaline earth metal salt thereof is present as polyanion.

11. Electroluminescent assemblies according to claim 1, characterized in that a photoluminescent material (component C) is present.

12. Electroluminescent assemblies according to claim 1, characterized in that substances which display photoluminescence, metal complexes, chelates or inorganic nanosize particles are present.

13. Electroluminescent assemblies according to claim 1, characterized in that they contain at least one compound selected from the group consisting of stilbenes, distilbenes, methine dyes, coumarins, naphthalimides, perylenes, rubrene, quinacridones, phenanthrenes, anthracenes, phthalocyanines, metal complexes of monovalent, divalent or trivalent metals which form chelates, inorganic nanosize particles.

14. Electroluminescent assemblies according to claim 12, characterized in that the metal is selected from the group consisting of lithium, sodium, potassium, magnesium, calcium, boron, aluminium, gallium, indium, rare earths and the inorganic nanosize particles are selected from the group consisting of CdS, CdSe, ZnS or ZnO.

15. Electroluminescent assemblies according to claim 1, characterized in that an oxine complex (8-hydroxyquinoline complex) of Al³⁺, Mg²⁺, In³⁺, Ga³⁺, Zn²⁺, Be²⁺, Li⁺, Ca²⁺, Na⁺ or tris(5-methyloxine)aluminium, tris(5-chloroquinoline)gallium or rare earth metals is present.

16. Electroluminescent assemblies according to claim 1, characterized in that a metal complex selected from among 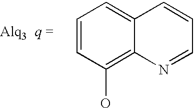
Inq₃, Gaq₃, Znq₂, Beq₂, Mgq₂,
or Al(qa)₃, Ga(qa)₃, In(qa)₃, Zn(qa)₂, Be(qa)₂, Mg(qa)₂ is present,
where [chemical structure]
